(19) United States
(12) Patent Application Publication
(10) Pub. No.: US 2005/0154309 A1
(43) Pub. Date: Jul. 14, 2005

(54) Medical device inline degasser

(75) Inventors: Tim Etchells, Bothell, WA (US); Gregory Paul Darlington, Snohomoish, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: LIPOSONIX, INC., Bothell, WA (US)

(21) Appl. No.: 10/750,370

(22) Filed: Dec. 30, 2003

(51) Int. Cl. A61B 8/14

(52) U.S. Cl. 600/459

(57) ABSTRACT

An apparatus for degassing a coupling fluid for use with a high intensity ultrasound system is disclosed. The apparatus has a fluid circuit having a vacuum chamber, a circulating pump, a vacuum pump and optional sensors for detecting one or more fluid parameters, such as dissolved gases. The coupling fluid may also serve as a coolant for a HIFU transducer.

### CROSS-REFERENCES TO RELATED APPLICATIONS

The subject matter of the present application is related to that of the following applications each of which is being filed on the same day as the present application: Ser. No. 10/______, entitled “Articulating Arm for Medical Procedures” (Attorney Docket No. 02356-000600US); Ser. No. 10/______, entitled “Disposable Transducer Seal” (Attorney Docket No. 02356-000700US); Ser. No. 10/______, entitled “Acoustic Gel with Dopant” (Attorney Docket No. 02356-000800US); 60/______, entitled “Position Tracking Device” (Attorney Docket No. 021356-000900US); 60/______, entitled “Method for Planning and Performing Ultrasound Therapy” (Attorney Docket No. 021356-001000US); 60/______, entitled “Ultrasound Therapy with Hood Movement Control” (Attorney Docket No. 021356-001100US); 60/______, entitled “Systems and Methods for the Destruction of Adipose Tissue” (Attorney Docket No. 021356-001200US); 60/______, entitled “Component Ultrasound Transducer” (Attorney Docket No. 021356-001300US); the full disclosure of each of these applications are incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

FIELD OF THE INVENTION

The present invention relates to an apparatus and method for removal of dissolved gasses in a coupling fluid used in high intensity focused ultrasound medical procedures.

During high intensity focused ultrasound (HIFU) procedures there is often a need to cool the face of the transducer or a patient's skin with a medium that will pass ultrasound energy with little or no attenuation or adverse effect. Typically this medium is water held within a transmission cavity with a cap or membrane, and through which the ultrasound energy passes.

One major issue with such a system arises from bubble formation caused by dissolved gasses being drawn out of solution. These bubbles provide an impedance mismatch to the ultrasound which causes reflections and localized heating, leading to observed effects such as reduced effectiveness of therapy, surface heating leading to the destruction of the cap or seal, or patient skin burns.

Atmospheric water for example, contain approximately 8.5 PPM (parts per million) O₂, and 14.5 PPM N₂ as well as other dissolved gasses. Using dissolved oxygen (DO) as an indicator (by partial pressures the relative contents of other gasses, CO₂, CO, N₂, etc . . . can be calculated) it is necessary to reduce the DO to less than 5 PPM in order to reduce the attenuation effects to a manageable level.

The common method used by the industry is to prepare the fluid by passing it through a filtration and de-ionisation process to remove impurities and particulates which may precipitate out, contaminate or provide nucleation sites for bubbles. The coupling fluid is then degassed to some minimum level before introduced into the system. Typically degassing is performed by bulk cavitation under a vacuum (e.g. using a Nold apparatus or the like) or boiling at atmospheric or sub atmospheric pressure and then sealing the degassed fluid in a container.

In a completely sealed system the dissolved gas content will remain constant, but as described below the gas content will strive to meet equilibrium with the partial pressure of the local atmospheric conditions. During short procedures or low power ultrasound procedures the re-gas rate is usually slow enough not to cause problems. In longer procedures and/or at higher powers, the probability that re-dissolved gas will be drawn into the fluid, and subsequently interfere with ultrasound transmission, goes up considerably since it is impossible to prevent gas diffusing through the system lining, joints and seals without investing in prohibitively expensive parts and materials.

The methods by which gasses come out of solution or enter the cooling system are various, some examples of the more common range from pressure changes within the cooling system caused by physical restriction or atmospheric conditions. Local pressure changes such as rectified diffusion from HIFU or temperature changes will bring gas out of solution as will displacement of the partial pressure of one gas by another, or by material leaching. Other methods by which gas may enter the system include diffusion through the tubing, seals and structure of the cooling system in the same way a balloon deflates, trapping micro bubbles within the surface structure and pockets of the cooling system, chemical reactions between materials in the cooling system, or as a by product of bacterial growth within the cooling system.

Precautions such as using low permeability materials for the tubing are regularly employed, but even with such precautions, the re-gas rate can become a major issue. Other methods used to reduce the effects of re-gassing include the introduction of surfactants or wetting agents to prevent bubble formation, using larger volumes of fluids, and the use of hydrophilic and/or hydrophobic polymers such as Polyvinaylpyyolidone (PVP). Experimental testing has shown these provide only a short term solution.

Numerous examples in the prior art show differing solutions to the problems of dealing with coupling HIFU transducers to a patient as well as providing an apparatus for degassing a fluid. However there has been thus far nothing demonstrating the feasibility or utility of an in line degassing mechanism combined with a HIFU therapy system during an actual medical procedure or application.

The inability of the prior art to maintain a controlled dissolved gas content in a cooling fluid over a prolonged procedure acts as a forced limitation to prolonged HIFU therapy.

Thus there remains a need for a degassed coupling fluid for use in a HIFU procedure to provide continuous or on demand degassing of the coupling fluid.

There is further a need for a way to prevent a HIFU transducer from over heating during a HIFU procedure.

There is still further a need for real time monitoring of dissolved gasses in a coupling fluid to ensure proper ultrasound transmission.

### BRIEF SUMMARY OF THE INVENTION

Thus an object of the present invention is to provide a system for degassing an ultrasound coupling fluid.

Another object of the present invention is to provide a cooling system for a HIFU transducer.

Still another object is to provide an integrated system for allowing the cooling of a HIFU transducer and a degassed coupling fluid in a single solution.

At least some of these objectives are met in a system and method for coupling a high intensity focused or other ultrasound transducer to a patient comprising a circuit for conveying a coupling fluid, the circuit having a circulation pump, a vacuum chamber connected to the circuit such that dissolved gasses are drawn out of the coupling fluid across a pressure gradient within the vacuum chamber, the pressure gradient created by a vacuum pump and a coupling reservoir connected to the circuit for coupling a transducer to a patient.

In a second aspect of the present invention, a chiller is added to the circuit whereby the coupling fluid can be refrigerated, and serve as a cooling fluid for the transducer.

In a third aspect of the present invention, there is an apparatus for performing high intensity focused ultrasound (HIFU) procedures, the apparatus comprising a first housing having a high intensity focused ultrasound (HIFU) transducer, a second housing having system electronics for controlling said transducer, a user interface, a display and a power supply, and a system for circulating a degassed coupling fluid wherein the coupling fluid is circulated between the first housing and the second housing, and the first housing and the second housing are in electronic communication with each other

The degassed coupling fluid is preferably re-circulated, however optionally the fluid may be continuously circulated from an external supply through the vacuum chamber to the reservoir and then exhausted from the system or apparatus.

### BRIEF DESCRIPTION OF THE DRAWINGS

FIG. 1 illustrates a schematic of fluid circuit for coupling a transducer.

FIGS. 2 and 2A illustrate alternative embodiments of a vacuum chamber.

FIGS. 3 and 3A illustrate alternative cross sections of a vacuum chamber.

FIG. 4 illustrates a transducer housing utilizing a degassing system.

### DETAILED DESCRIPTION OF THE INVENTION

In a principle embodiment of the invention, there is a system for coupling a high intensity focused ultrasound transducer to a patient. The system comprises a circuit for conveying a coupling fluid, the circuit usually has a circulation pump. A vacuum chamber or other reduced pressure environment is connected to the circuit such that dissolved gasses are drawn out or “degassed” from the coupling fluid across a pressure gradient within the vacuum chamber. The pressure gradient is created by (and maintained by) a vacuum pump or other vacuum source. There is also a coupling reservoir connected to the circuit for coupling a transducer to a patient.

In an alternative embodiment, there is an apparatus for performing high intensity focused ultrasound (HIFU) procedures. The apparatus comprises a first housing having a high intensity focused ultrasound (HIFU) transducer, and a second housing having system electronics for controlling the transducer, a user interface, a display and a power supply. A system for circulating a degassed coupling fluid is used to couple the transducer to a patient wherein the coupling fluid is circulated between the first housing and the second housing. The first and second housing also are in electrical communication.

The apparatus described is an inline degassing device to continuously degas the coupling fluid for therapeutic ultrasound use. The coupling fluid may be any low viscosity fluid suitable for such applications. Although mineral oils or hydro-gels are used as coupling agents, the preferred fluids for use with the present invention are aqueous solution or pure water. Mineral oils, hydro-gels and the like may still be used to couple the transducer to the patient, however they would be additional to the fluid used with the present invention. Water based solutions may contain additional impurities designed to prevent or inhibit the grown of bacteria, algae or fungus within the circulation circuit and its components. It is possible to use non-water based fluid as well, however these are not part of the preferred embodiment. Regardless of the fluid used as a coupling agent, the system of the present invention allows the amount of dissolved gas in the fluid to be controlled to within acceptable levels and even in a dynamic nature if required. An inline technique has several benefits, the need for frequent fluid replacement is reduced, the need for expedience in replacing caps or seals is reduced and the effectiveness of therapy is increased (because the fluid, cap or seal does not degrade over the treatment duration).

The vacuum chamber follows understood principles and methods used in degassing water and other fluids. In brief, the system uses a semi-permeable membrane or barrier of sufficient size and hydrophobic properties to pass gas molecules but not coupling fluid molecules, in conjunction with a pressure gradient (vacuum chamber) to draw the dissolved gasses out of solution and through the barrier. The use of multiple tubes to increase the surface area, allow an increase in the degas rate. By varying the level of vacuum, the re-gas rate as seen at a high intensity ultrasound transducer can be strictly controlled. The term vacuum chamber should be understood to mean a chamber wherein gas is drawn from solution using any number of well understood methods and articles within the chamber. In a simple embodiment, the coupling agent may pass through a series of tubes, relying on the pressure gradient across the tube membranes to draw out dissolved gasses. In a more complex system the coupling agent may be pressurized into a series of baffles in combination with a low pressure gradient across the baffle membranes, and high heat of the coupling agent. All of which would facilitate the removal of gases from solution.

The system for degassing a fluid for use with an ultrasound system has a reservoir serving to couple a transducer and a patient. The reservoir need not completely surround or engulf the transducer so long as it at least covers the face of the transducer so the reservoir can serve as an acoustic coupling device between a transducer and the desired target. The reservoir may also be designed to incorporate a transducer within, so that the coupling fluid completely surrounds the transducer's lens. Whether the reservoir surrounds the transducer or provides a coupling buffer to the transducer, the reservoir is connected to the circuit such that the coupling fluid can flow into and out of the reservoir. Preferably the inflow and exhaust of the fluid are on opposing sides of the reservoir to ensure the fluid is regularly circulated and does not form stagnant pools within the reservoir. In addition it would be preferable to have flow valves for controlling the flow of the coupling fluid into the reservoir. The flow into the reservoir can be temporarily halted if an element of the reservoir needs to be replaced.

The system also has a vacuum chamber for degassing the coupling fluid. The vacuum chamber has one or more gas permeable membranes through which gasses may be drawn from the coupling fluid. A circulation pump is needed for circulating the coupling fluid through the vacuum chamber and the reservoir. One or more optional sensors can be used to measure the dissolved gas content of the coupling fluid, the flow rate and/or the temperature of the coupling fluid. A vacuum pump is used for extracting dissolved gases from the coupling fluid such that the coupling fluid has a dissolved oxygen content of 5 PPM or less.

In an alternative embodiment the coupling agent may be divided into a plurality of flow tubes. One flow tube can pass through the vacuum chamber while a second flow tube can by pass the vacuum chamber. In this manner it is not necessary to degas the entire volume of the coupling fluid during each cycle of the fluid through the circuit. Where the fluid has obtained a desired level of dissolved gas, the partial degassing of coupling fluid can maintain the low dissolved gas levels without expending the energy needed to de-gas the entire volume. The second flow tube can pass through an additional component, such as a chiller or other apparatus before being combined with the coupling fluid exiting from the vacuum chamber. Where the fluid is sufficiently degassed and the re-gas rate of the coupling fluid is below acceptable gas concentration levels, the vacuum chamber may be inactivated. In this manner fluid passes through the vacuum chamber unaffected, or is completely rerouted around the vacuum chamber.

An alternative embodiment of the invention provides for sensors integrated into the closed circuit to facilitate in the control of the coupling fluid. In this case a system for circulating a degassed ultrasound coupling fluid comprises a closed circuit for conveying the fluid, a circulation pump for circulating the fluid through the circuit, a vacuum chamber connected to the circuit and connected to a vacuum pump for producing a pressure gradient within the vacuum chamber, a reservoir for coupling a transducer to a patient, and one or more sensors in electronic communication with a controller such that the controller can affect the circulation pump or the vacuum pump in response to information received from the sensors.

Various sensors can be utilized to measure dissolved gases, flow rate, temperature, impurities within the coupling fluid, and any other parameter of the coupling fluid. The sensors can provide measurements to the controller, and thus the controller can adapt to the data received from the sensors to alter any of the systems functions until the sensor reports data within the desired specification of the controller. The sensors may be placed anywhere along the fluid circuit. In this way the system can operate on a duty cycle off and on for both circulation and for degassing. The cycles can be integrated so that circulation and degassing coincide, or they can operate independently. Among the gases, dissolved oxygen is the preferred gas to measure. Using Daltons Law and Henry's Law it is possible to calculate the partial pressures of other dissolved gasses in solution. By removing gases until the dissolved oxygen content drops below 5 PPM, it is possible to determine the total dissolved gas content. If the dissolved Oxygen is at or below 5 PPM the attenuation of ultrasound signal during a HIFU procedure becomes manageable. It is apparent then that the lower the dissolved gas content of the coupling fluid, the better and that a 0 PPM value is ultimately desirable. Using the system of the present invention acceptable levels of dissolved oxygen may approach 1 PPM, providing a substantial safety margin in dissolved gas content of the coupling fluid.

In either embodiment, the circulation of the coupling fluid either around the transducer or across the face of the transducer has an added benefit in the form of preventing the transducer from becoming too hot during a procedure. The addition of a chiller to the system can further provide for a temperature control of the transducer if desired. In this manner the coupling fluid also serves as a cooling fluid.

Referring now to the drawings, there is a vacuum chamber 706 for degassing the coupling fluid and the chamber may be of a variety of known designs. Since the apparatus 7 is a component of a larger system, preservation of the available space is preferred. Thus the vacuum chamber 706 is preferably small with a number of baffles contained within the vacuum chamber 706 to maximize the surface area the coupling fluid is exposed to. By using thin diameter tubes 713, panels or coils, the coupling fluid may be flushed through the vacuum chamber volume 724 and exposed to the gas permeable membrane 730 with a favorable volume to area ratio. Furthermore this ratio can double as a cooling baffle for the coupling fluid. As the coupling fluid moves through the vacuum chamber 706, the pressure gradient inside the chamber draws dissolved gasses out of solution and those gases diffuse across the membrane into the lower pressure volumes of the vacuum chamber volume 724. A vacuum source, such as a vacuum pump 704, is used to maintain the negative pressure either continuously or based on a duty cycle tied to a timer (not shown), or a sensor 714 for detecting the presence of dissolved gases. Alternatively, in hospitals and other locations where vacuum systems are provided, these systems can be connected to the vacuum chamber to provide the desired negative pressure. The flow rate of the coupling fluid through the vacuum chamber 706 is regulated to ensure sufficient exposure of the coupling fluid to the membrane and pressure gradient 724. The flow rate can also be controlled in order to increase the residence time in the baffles if increased heat exchange is desired. If the flow rate and dissolved gas levels are sufficient so that a reduced volume of the coupling fluid can be degassed to maintain the desired levels, some portion of the coupling fluid may be redirected through a control valve 724 into a bypass tube 718. The coupling fluid that is directed into the bypass tube 718 will not be degassed.

The circulation pump 702 insures the coupling fluid is constantly circulated through the fluid circuit 712 whether or not the vacuum pump 704 is active. A chiller 710 may be added as part of the apparatus to extract heat from the coupling fluid as it circulates. The chiller 710 may be part of the ultrasound system or part of the degassing apparatus. In one alternate embodiment there is a chiller or temperature control means for maintaining a constant temperature in the vacuum chamber 706. Thus when the coupling fluid moves through the vacuum chamber 706 and experiences the high surface to volume ratio, heat is effectively drawn away from the cooling solution and evacuated along with the gas.

As an option, a chiller 710 may be placed in the circulation path prior to the transducer cooling chamber 502, to ensure the coupling fluid is at the desired temperature for cooling the transducer 600, or it may be on the exhaust side of the transducer cooling chamber 502 so there is less temperature discomfort for the patient.

Another optional element is a storage tank 708 for holding a reserve amount of coupling fluid. The storage tank 708 may also act as a heat sink due to a larger thermal mass. The storage tank 708 has intake 720 and exhaust 722 lines for the introduction of the coupling fluid, and for removing the coupling fluid from the system. The intake 720 and exhaust 722 lines are sealed during normal operation, but can be opened during a HIFU procedure if necessary to allow the introduction of additional coupling fluid, or the removal of coupling fluid.

A membrane can be integrated into the storage tank to reduce minor fluctuations in both pressure and volume in the coupling fluid degassing circuit. The membrane forms a seal with the coupling fluid within the storage tank and can fluctuate with minor changes in the pressure and volume of the fluid. In this manner if the pressure or volume of the fluid in the system changes slightly, the membrane can flex with the fluid in the storage tank. This allows the coupling agent through out the system to operate continuously with a volume in the system circuit that matches minute changes in the volume and pressure of the coupling fluid itself. Alternatively, the pressure variable membrane may be replaced by a device having an adaptive volume somewhere else in the system. An example would be the placement of a fluid tight balloon or syringe attached to the system exhaust valve.

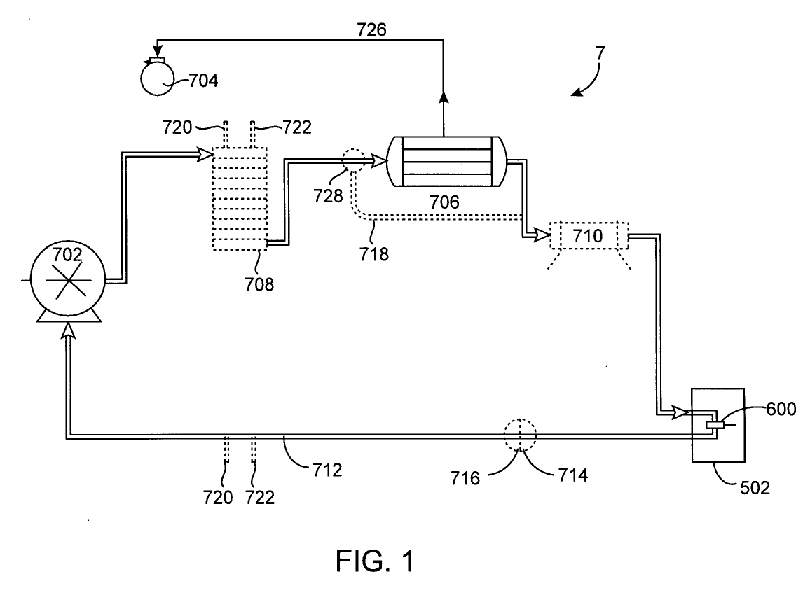

The principle embodiment of the invention is shown in FIG. 1. The fluid circuit 712 is shown as a schematic. There is a circulation pump 702, a vacuum chamber 706 and a reservoir 502 surrounding an ultrasound transducer 600. Following the coupling fluid through the apparatus 7, the coupling fluid is first introduced into the circuit through an intake line 720 somewhere in the system. The intake 720 may be in the storage tank 708, or it may be anywhere along the circuit line. Once the coupling fluid is introduced into the system, degassing begins. The circulation pump 702 is activated so the coupling fluid circulates through the entire system and through the vacuum chamber 706 to remove dissolved gasses within the coupling fluid. Preferably the intake is air tight and provides a means for the coupling fluid to be introduced into the HIFU system ready for immediate use. Alternatively, a suitable fluid may be introduced into the system and degassed through the vacuum chamber 706 prior to use.

The coupling fluid then flows into the transducer cooling reservoir 502, around the transducer 600, and back to the circulation pump 702. Any suitable pump may be used so long as the integrity of the system is maintained and the flow rate and gas concentrations are controllable. The coupling fluid passes from the circulation pump 702 to either an optional storage tank 708 or directly to the vacuum chamber 706. If there is a storage tank 708 the fluid may be chilled here, or it may simply be a reservoir to ensure there is sufficient fluid in the system. The vacuum chamber 706 is where the degassing actually takes place. As previously described the coupling fluid passes through a plurality of conduits shaped to maximize the surface area to the vacuum volume. The membrane 730 separating the coupling fluid from the vacuum chamber 706 is gas permeable but not fluid permeable (not permeable to the coupling fluid). A pressure gradient (such as a negative pressure within the vacuum chamber) is maintained in the vacuum chamber 706 to ensure that dissolved gases are continuously removed from the coupling fluid and are transported out and away from the coupling fluid. Depending on the efficiency of the system, both the vacuum pump and circulation pump may be operated full time, or based on a duty cycle. The operation of the circulation pump and vacuum pump can also be independent of the other.

Although the apparatus has been detailed and drawn in a particular order of parts, the ordering of components along the system is not critical and various components may be moved in relation to other components with out departing from the spirit of the invention. Nothing regarding the ordering of the components of the apparatus should be taken in a limiting sense.

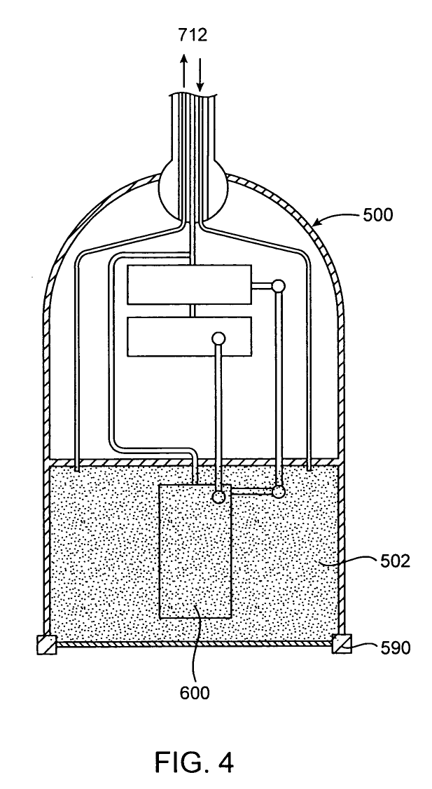

In yet another embodiment, we describe an apparatus for performing high intensity focused ultrasound (HIFU) procedures, the apparatus comprising a first housing having a high intensity focused ultrasound (HIFU) transducer (see FIG. 4), and a second housing (not shown) having system electronics for controlling the transducer, a user interface, a display and a power supply, and a system for circulating a degassed coupling fluid wherein the coupling fluid is circulated between the first and second housing. In this embodiment, the bulk of the inline degassing system is in the second housing, while only the reservoir 502 is in the first housing along with the transducer. The coupling fluid circuit 712 enters and exits the first housing at a common point preferably where the system electronics communicates with the transducer 600 and any other devices within the first housing. The coupling fluid flows into a reservoir 502 and serves as a coupling agent for the transducer. The coupling fluid can completely surround the transducer as shown, or fill a separate cushion or sack used to act as a coupling device. The various sensors can be placed in either the first or second housing if desired so long as they are still on the fluid circuit.

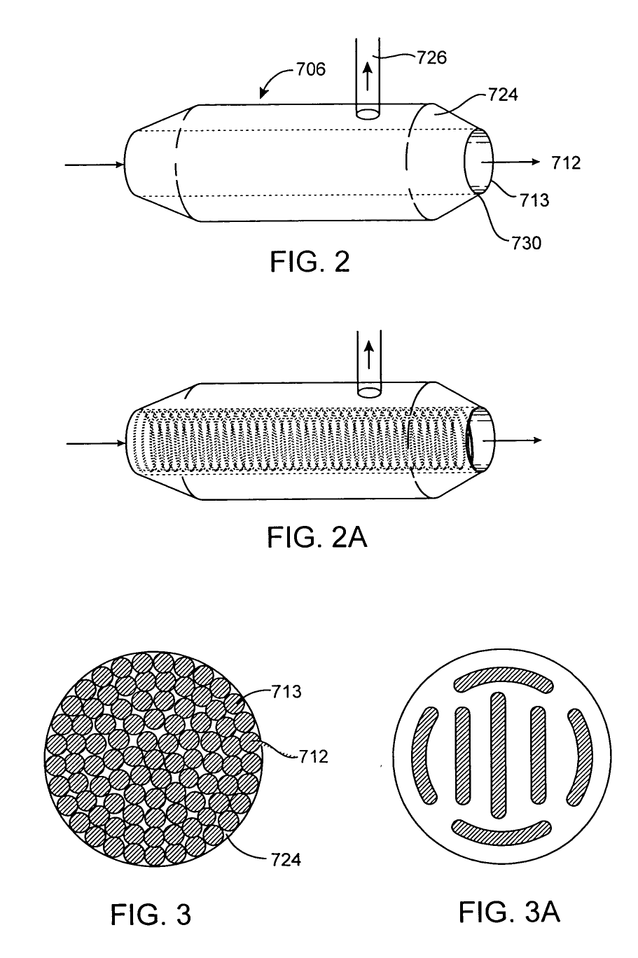

The efficiency of the vacuum chamber 706 depends on several factors including the residence time of the coupling fluid in the vacuum chamber and the ability of the dissolved gases to come out of solution. It is desirable then to increase the exposure of the coupling fluid to the pressure gradient of the vacuum chamber. The coupling fluid can pass through the vacuum chamber 706 through a single gas permeable membrane conduit 730 (FIG. 2) and exposing the fluid near the membrane of the conduit to the pressure gradient in the vacuum space 724. The flow direction is indicated by the arrows indicating the fluid is still inline with the fluid circuit 712. Gas removed from solution is vented through a conduit 726 toward the vacuum pump 704. Alternatively the fluid may pass through a series of low diameter gas permeable tubes (FIG. 3) or through one or more tortuous tubes, such as a coil, to increase residence time in the chamber (FIG. 2A). Alternatively the fluid may be pumped through a series of baffles (FIG. 3A). The embodiments described and shown are merely illustrative and not meant to be limiting in any sense.

One or more sensors 714, 716 may be used to measure various parameters of the coupling fluid. Parameters that may be measured include the amount of dissolved gasses, the flow rate, the fluid temperature, electrical conductivity or any other parameter desired. The data may be relayed to the system electronics, which can then adjust the flow rate or pressure gradient of the vacuum chamber. Alternatively an independent electronic controller (not shown) can be incorporated into the system for direct feedback and control of the system operations.

In application, the coupling fluid is introduced to the apparatus of the present invention when the ultrasound system is either off or idle (the ultrasound system may be on, however the transducer is not active). The vacuum and circulation pumps are activated (though a single pump can be used for both functions) so the coupling fluid circulates throughout the apparatus. The vacuum pump creates the negative pressure in the chamber and as the coupling fluid flows through the chamber at a controlled speed the gas is drawn out of solution across a gas permneable membrane. The degassed coupling fluid is then circulated around a transducer through a reservoir.

If the system has a storage tank, the system can be filled through an input valve attached to the storage tank. If a balloon or diaphragm is used as a means for reducing fluctuations in the pressure and volume of the fluid in the system, one must exercise care not to over fill or under fill the fluid circuit such that the balloon or diaphragm are no longer able to compensate for small pressure and volume changes in the system.

In addition, a filter can be introduced into the system anywhere along the fluid circuit to remove particulate matter. Reduction of particulate matter is desirable as it decreases the likelihood of nucleation occurring, and thus reduces or prevents bubble formation in the coupling fluid.

The apparatus may be used as a sealed system with the introduction of the cooling fluid done once prior to an ultrasound procedure being performed. However if the degassing efficiency is sufficient to keep the coupling fluid at or below 5 PPM of dissolved oxygen, the system may be run continuously and the coupling fluid may be continuously supplied (as from a tap water line) and then evacuated from the system.

1. A system for coupling a high intensity focused ultrasound transducer to a patient comprising:
a circuit for conveying a coupling fluid;
pump for circulating coupling fluid through the circuit;
a vacuum chamber connected to apply a pressure gradient to said circuit such that dissolved gasses are drawn out of said coupling fluid; and
a coupling reservoir connected to said circuit for coupling a transducer to a patient.

2. The system as described in claim 1 further comprising a chiller on said circuit for cooling said coupling fluid.

3. The system as described in claim 1, wherein said vacuum chamber comprises one or more gas permeable membranes through which gas may be drawn from said fluid.

4. The system as described in claim 1, wherein the pump is also connected to draw a vacuum in the vacuum chamber.

5. The system as described in claim 1, further comprising a control device for adjusting said circulation pump.

6. The system as described in claim 1, further comprising one or more sensors.

7. The system of claim 6, wherein said one or more sensors are in electronic communication with said control device.

8. The system as described in claim 6, wherein said sensor(s) capable of reporting information to an operator.

9. The system of claim 1, wherein said fluid is a coolant for controlling the temperature of a transducer.

10. The system of claim 1, further comprising a storage tank located on said circuit.

11. The system of claim 1, further comprising a means for reducing pressure and volume fluctuations.

12. The system as described in claim 1, having one or more valves in said circuit.

13. The system as described in claim 1, wherein said fluid is water.

14. An apparatus for performing high intensity focused ultrasound (HIFU) procedures, the apparatus comprising:
a first housing having a high intensity focused ultrasound (HIFU) transducer;
a second housing having system electronics for controlling said transducer, a user interface, a display and a power supply; and
a system for circulating a degassed coupling fluid wherein said coupling fluid is circulated between said first housing and said second housing and said first housing and said second housing are in electronic communication.

15. The apparatus as described in claim 14, wherein said system for circulating a degassed coupling fluid further comprises one or more sensors in electronic communication with said system electronics, and said system electronics able to operate as a controller for said system for circulating a degassed coupling fluid.

16. The apparatus as described in claim 14, wherein the system for circulating a degassed coupling fluid is a cooling system for dissipating heat from said high intensity focused ultrasound (HIFU) transducer.

17. A method for performing ultrasound therapy, said method comprising:
applying an ultrasound transducer to a tissue surface of the patient;
energizing the transducer to emit ultrasonic energy to the patient;
circulating a coupling fluid to the transducer; and
applying a vacuum to the circulating coupling fluid to degas said fluid.
